(19) United States
(12) Patent Application Publication
(10) Pub. No.: US 2022/0401414 A1
(43) Pub. Date: Dec. 22, 2022

(54) TREATMENT AND PREVENTION OF A NEURODEGENERATIVE DISORDER

(71) Applicant: Inflazome Limited, Dublin 2 (IE)

(72) Inventors: Matthew COOPER, Cambridge (GB); Luke Anthony John O'NEILL, Dublin 2 (IE)

(21) Appl. No.: 17/775,113

(22) PCT Filed: Nov. 6, 2020
(86) PCT No.: PCT/EP2020/081263

§ 371 Date: May 6, 2022

(30) Foreign Application Priority Data
Nov. 7, 2019 (GB) 1916236.1
Jan. 20, 2020 (GB) 2000805.8
Mar. 13, 2020 (GB) 2003642.2

(51) Int. Cl. A61K 31/415; A61P 25/28; A61P 25/16; A61K 9/00

(52) U.S. Cl. CPC A61K 31/415; A61P 25/28; A61P 25/16; A61K 9/0053

(57) ABSTRACT

The present invention relates to a compound of formula (I): for use in the treatment or prevention of a neurodegenerative disorder.

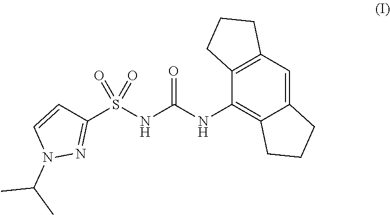 [SMILES: CC(C)n1ccc(S(=O)(=O)NC(=O)Nc2c3c(cc4c2CCC4)CCC3)n1]

The present invention relates to a compound of formula (I):

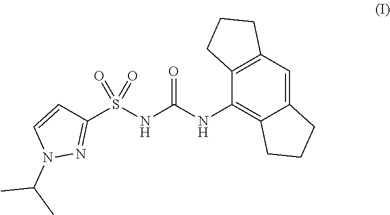 [SMILES: CC(C)n1ccc(S(=O)(=O)NC(=O)Nc2c3c(cc4c2CCC4)CCC3)n1]

for use in the treatment or prevention of a neurodegenerative disorder.

Neurodegenerative disorders include Parkinson's disease, Alzheimer's disease, Motor Neurone disease (Amyotrophic Lateral Sclerosis), Multiple System Atrophy, Progressive Supranuclear Palsy, Frontotemporal Dementia, Huntington's disease, Ataxia and Neurodegenerative Prion Diseases.

Parkinson's disease (PD) is a chronic progressive neurodegenerative disorder caused by the death of key cells in the brain, leading to the loss of dopamine, a chemical used to control the movements a person makes as well as emotional responses. While symptoms can be controlled by levodopa therapy over a few years, the disease is still progressing and no disease-modifying treatments are currently available. Targeting neuroinflammation through inhibiting NLRP3 addresses this major unmet medical need.

Approximately 30 million people globally have Alzheimer's disease (AD) for which there is no known cure. The pathogenesis of Alzheimer's disease is widely believed to be driven by the production and deposition of the amyloid-β peptide (Aβ) which has been shown to drive neuroinflammation and subsequently neuronal death and disease progression involving NLRP3 activation.

This invention is based in part on the discovery that the compound of formula (I) is particularly effective in crossing the blood-brain barrier and in inhibiting the NLRP3 inflammatory response in microglia, thus providing effective treatment of neurodegenerative disorders such as Parkinson's disease and Alzheimer's disease. Most especially, neuroinflammation arising from such disorders may be effectively inhibited by the oral administration of the compound of formula (I).

In a first aspect of the present invention, there is provided a compound of formula (I):

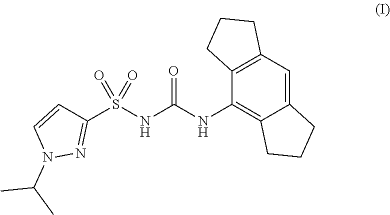 [SMILES: CC(C)n1ccc(S(=O)(=O)NC(=O)Nc2c3c(cc4c2CCC4)CCC3)n1]

or a pharmaceutically acceptable salt thereof, for use in the treatment or prevention of a neurodegenerative disorder.

In one embodiment, the neurodegenerative disorder is Parkinson's disease. In another embodiment, the neurodegenerative disorder is Alzheimer's disease. In another embodiment, the neurodegenerative disorder is Motor Neurone disease. In another embodiment, the neurodegenerative disorder is Huntington's disease. In another embodiment, the neurodegenerative disorder is Multiple System Atrophy. In another embodiment, the neurodegenerative disorder is Progressive Supranuclear Palsy. In another embodiment, the neurodegenerative disorder is Frontotemporal Dementia. In another embodiment, the neurodegenerative disorder is Ataxia, such as a Spinocerebellar Ataxia (SCA). In another embodiment, the neurodegenerative disorder is a Neurodegenerative Prion Disease, such as Creutzfeldt-Jacob Disease (CJD), variant CJD, bovine spongiform encephalopathy (BSE) or scrapie.

In one embodiment, the treatment or prevention comprises the treatment or prevention of neuroinflammation. Typically, the treatment or prevention of neuroinflammation is achieved via NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

In one embodiment, the treatment or prevention comprises the oral administration of the compound or the salt thereof. In a further embodiment, the treatment or prevention comprises the once daily oral administration of the compound or the salt thereof.

In another embodiment, the compound or the salt thereof is for use in the prevention of motor loss in a patient suffering from a neurodegenerative disorder. The neurodegenerative disorder may be any of those listed above. Typically, the compound or the salt thereof is for use in the prevention of motor loss in a patient suffering from Parkinson's disease, most typically wherein the use comprises the oral administration of the compound or the salt thereof. In one embodiment, the compound or the salt io thereof is administered prior to the onset of motor loss.

In a further embodiment, the compound or the salt thereof is for use in the reduction of motor loss in a patient suffering from a neurodegenerative disorder. The neurodegenerative disorder may be any of those listed above. Typically, the compound is or the salt thereof is for use in the reduction of motor loss in a patient suffering from Parkinson's disease, most typically wherein the use comprises the oral administration of the compound or the salt thereof.

In one embodiment, the compound or the salt thereof is for use in the prevention of dopaminergic degeneration in a patient suffering from a neurodegenerative disorder.

The neurodegenerative disorder may be any of those listed above. Typically, the compound or the salt thereof is for use in the prevention of dopaminergic degeneration in a patient suffering from Parkinson's disease, most typically wherein the use comprises the oral administration of the compound or the salt thereof.

In a further embodiment, the compound or the salt thereof is for use in slowing, halting or reversing a decrease in dopamine levels in a patient suffering from a neurodegenerative disorder. The neurodegenerative disorder may be any of those listed above. Typically, the compound or the salt thereof is for use in slowing or halting a decrease in dopamine levels. More typically, the compound or the salt thereof is for use in slowing a decrease in dopamine levels. In one embodiment, the compound or the salt thereof is for use in slowing, halting or reversing a decrease in dopamine levels in a patient suffering from Parkinson's disease, typically wherein the use comprises the oral administration of the compound or the salt thereof. Typically, the compound or the salt thereof is for use in slowing or halting a decrease in dopamine levels in a patient suffering from Parkinson's disease. More typically, the compound or the salt thereof is for use in slowing a decrease in dopamine levels in a patient suffering from Parkinson's disease.

In one embodiment, the compound or salt is a sodium salt, such as a monosodium salt. In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3°20, 8.7°20, and 20.6°20, all ±0.2°20. In one embodiment, the crystalline monosodium monohydrate salt has an XRPD spectrum in which the 10 most intense peaks include 5 or more peaks which have a 20 value selected from: 4.3°2θ, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

Typically, in accordance with any embodiment of the first aspect of the invention, the treatment or prevention comprises the administration of the compound or the salt thereof to a patient. The patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

In a second aspect of the present invention, there is provided a pharmaceutical composition comprising a pharmaceutically acceptable excipient and a compound or salt of the first aspect of the present invention. In one embodiment, the pharmaceutical composition is suitable for oral administration.

In a third aspect of the present invention, there is provided a method for the treatment or prevention of a neurodegenerative disorder in a patient in need thereof, wherein the method comprises administering to the patient in need thereof a therapeutically or prophylactically effective amount of a compound of formula (I):

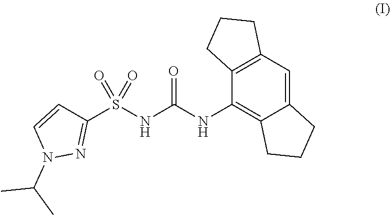 [SMILES: CC(C)n1ccc(S(=O)(=O)NC(=O)Nc2c3c(cc4c2CCC4)CCC3)n1]

or a pharmaceutically acceptable salt thereof.

In one embodiment, the neurodegenerative disorder is Parkinson's disease. In another embodiment, the neurodegenerative disorder is Alzheimer's disease. In another embodiment, the neurodegenerative disorder is Motor Neurone disease. In another embodiment, the neurodegenerative disorder is Huntington's disease. In another embodiment, the neurodegenerative disorder is Multiple System Atrophy. In another embodiment, the neurodegenerative disorder is Progressive Supranuclear Palsy. In another embodiment, the neurodegenerative disorder is Frontotemporal Dementia. In another embodiment, the neurodegenerative disorder is Ataxia, such as a Spinocerebellar Ataxia (SCA). In another embodiment, the neurodegenerative disorder is a Neurodegenerative Prion Disease, such as Creutzfeldt-Jacob Disease (CJD), variant CJD, bovine spongiform encephalopathy (BSE) or scrapie.

In one embodiment, the treatment or prevention comprises the treatment or prevention of neuroinflammation. Typically, the treatment or prevention of neuroinflammation is achieved via NLRP3 inhibition.

In one embodiment, the treatment or prevention comprises the oral administration of the compound or the salt thereof. In a further embodiment, the treatment or prevention comprises the once daily oral administration of the compound or the salt thereof.

In another embodiment, the method is for the prevention of motor loss in a patient suffering from a neurodegenerative disorder. The neurodegenerative disorder may be any of those listed above. Typically, the method is for the prevention of motor loss in a patient suffering from Parkinson's disease, most typically wherein the method comprises the oral administration of the compound or the salt thereof. In one embodiment, the compound or the salt thereof is administered prior to the onset of motor loss.

In a further embodiment, the method is for the reduction of motor loss in a patient suffering from a neurodegenerative disorder. The neurodegenerative disorder may be any of those listed above. Typically, the method is for the reduction of motor loss in a patient suffering from Parkinson's disease, most typically wherein the method comprises the oral administration of the compound or the salt thereof.

In one embodiment, the method is for the prevention of dopaminergic degeneration in a patient suffering from a neurodegenerative disorder. The neurodegenerative disorder may be any of those listed above. Typically, the method is for the prevention of dopaminergic degeneration in a patient suffering from Parkinson's disease, most typically wherein the method comprises the oral administration of the compound or the salt thereof.

In a further embodiment, the method is for slowing, halting or reversing a decrease in dopamine levels in a patient suffering from a neurodegenerative disorder. The neurodegenerative disorder may be any of those listed above. Typically, the method is for slowing or halting a decrease in dopamine levels. More typically, the method is for slowing a decrease in dopamine levels. In one embodiment, the method is for slowing, halting or reversing a decrease in dopamine levels in a patient suffering from Parkinson's disease, typically wherein the method comprises the oral administration of the compound or the salt thereof. Typically, the method is for slowing or halting a decrease in dopamine levels in a patient suffering from Parkinson's disease. More typically, the method is for slowing a decrease in dopamine levels in a patient suffering from Parkinson's disease.

In one embodiment, the compound or salt is a sodium salt, such as a monosodium salt. In one embodiment, the compound or salt is a monohydrate. In one embodiment, the compound or salt is crystalline. In one embodiment, the compound or salt is a crystalline monosodium monohydrate salt. In one embodiment, the crystalline monosodium monohydrate salt has an XRPD spectrum comprising peaks at: 4.3°2θ, 8.7°2θ, and 20.6°2θ, all ±0.2°2θ. In one embodiment, the crystalline monosodium monohydrate salt has an XRPD spectrum in which the 10 most intense peaks include 5 or more peaks which have a 2θ value selected from: 4.3°2θ, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ. The XRPD spectrum may be obtained as described in WO 2019/206871, which is incorporated in its entirety herein by reference.

In one embodiment, the crystalline monosodium monohydrate salt is as described in WO 2019/206871, which is incorporated in its entirety herein by reference. In one embodiment, the crystalline monosodium monohydrate salt has the polymorphic form described in WO 2019/206871, which is incorporated in its entirety herein by reference. In one embodiment, the crystalline monosodium monohydrate salt is prepared according to the method described in WO 2019/206871, which is incorporated in its entirety herein by reference.

In accordance with any embodiment of the third aspect of the invention, the patient may be any human or other animal. Typically, the patient is a mammal, more typically a human or a domesticated mammal such as a cow, pig, lamb, sheep, goat, horse, cat, dog, rabbit, mouse etc. Most typically, the patient is a human.

### Experimental

### FIGURES

FIG. 1: Study A—Levels of the compound of formula (I) in the MetaQuant dialysate from the left striatum of healthy freely-moving adult male mice following oral administration of 1 or 20 mg/kg of the compound (mean+SEM, n=4 per group).

FIG. 2: Study A—Levels of the compound of formula (I) in the MetaQuant dialysate from the right striatum of healthy freely-moving adult male mice following oral administration of 1 or 20 mg/kg of the compound (mean+SEM, n=4 per group).

FIG. 3: Study B - Levels of the compound of formula (I) in the MetaQuant dialysate from the left striatum of freely-moving adult male 6-OHDA mice following oral administration of 1 or 20 mg/kg of the compound (mean+SEM, n=4 per group).

FIG. 4: Study B—Levels of the compound of formula (I) in the MetaQuant dialysate from the right striatum of freely-moving adult male 6-OHDA mice following oral administration of 1 or 20 mg/kg of the compound (mean+SEM, n=4 per group).

FIG. 5: Study C—Oral treatment with the compound of formula (I) (CPD) protects against nigrostriatal dopaminergic degeneration in the 6-OHDA model of Parkinson's disease. A) Amphetamine-induced ipsilateral rotations quantified 21 days after 6-OHDA (12 μg) treatment showing that 3 mg/kg p.o. protects against dopaminergic degeneration (n=10 mice/group). B-D) Levels of striatal dopamine and its metabolites DOPAC and HVA in treated 6-OHDA mice demonstrating preservation of the striatal dopaminergic terminals with drug treatment (n=10 mice/group). Data represented as mean±S.E.M. *P<0.05, **P<0.01, *P<0.001, ****P<0.0001, by one-way analysis of variance (ANOVA), is and Tukey's post test.

FIG. 6: Study D—3 mg/kg oral treatment with the compound of formula (I) (CPD) protects against nigrostriatal dopaminergic degeneration in the 6-OHDA model of Parkinson's disease with greater efficacy than MCC950. A) Amphetamine-induced ipsilateral rotations quantified 21 days after 6-OHDA (12 μg) treatment showing that 3 mg/kg p.o. of the compound of formula (I) protects against dopaminergic degeneration compared with MCC950 (n=10 mice/group). B) Levels of striatal dopamine in 6-OHDA mice treated with the compound of formula (I) or MCC950, demonstrating preservation of the striatal dopaminergic terminals with drug treatment (n=10 mice/group). Data represented as mean±s.e.m. *P<0.05, **P<0.01, ***P<0.001, ****P<0.0001, by one-way ANOVA, and Tukey's post-test.

FIG. 7: Study E—The compound of formula (I) (CPD) displays higher potency than MCC950 in inhibiting NLRP3 inflammasome in primary microglia. A) Dose-dependent inhibition of ATP-induced NLRP3 inflammasome activation in primed microglia by the NLRP3 inhibitor MCC950. The IC₅₀ of MCC950 inhibition with ATP (5 mM) was determined to be 7.5 nM for primary mouse microglia. B) Dose-dependent inhibition of ATP-induced NLRP3 inflammasome activation in primed microglia by the compound of formula (I) (CPD). The IC₅₀ of inhibition with ATP (5 mM) for the compound of formula (I) was determined to be 4.74 nM for primary mouse microglia.

FIG. 8: Study F—Dose-dependent inhibition of ATP-induced NLRP3 inflammasome activation in primed healthy human microglia by the compound of formula (I) (CPD). The IC₅₀ of inhibition with ATP (5 mM) for the compound of formula (I) was determined to be 142 nM for primary human microglia isolated from one healthy donor. Data represent n=1 healthy donor and n=4 technical replicates. Error bars SEM.

FIG 9: Study G—Dose-dependent inhibition of ATP-induced NLRP3 inflammasome activation in primed Parkinson's disease human microglia by the compound of formula (I) (CPD). The IC₅₀ of inhibition with ATP (5 mM) for the compound of formula (I) was determined to be 101 nM for primary human microglia isolated from Parkinson's patients. Data represent n=3 Parkinson's donors and n=14 technical replicates. Error bars SEM.

FIG. 10: Study H—Oral treatment with the compound of formula (I) (CPD) protects against motor deficits in the PFF-Synuclein model of Parkinson's disease. A) Rotarod test in a-synuclein PFF-injected mice at 4, 6, 8 and 10 months after PFF-injections, prophylactic treatment with the compound of formula (I) in the drinking water (0.3 mg/ml) commencing 24 h before PFF-injections or therapeutic treatment commencing 4 months after injections (n=8-10 mice per group). PBS-injected mice were used as saline. B) Balance beam performance measured as time taken to cross the beam in a-synuclein PFF-injected mice at 4, 6, 8 and 10 months. PBS-injected mice were used as saline. *c.f Saline, #c.f PFF-Synuclein, Data are mean±SEM, ns=not significant, *#P<0.05,**##P<0.01,***###P<0.001****####P<0.0001, by one-way analysis of variance (ANOVA), and Tukey's post test.

FIG. 11: Study H—Oral dosing with the compound of formula (I) (CPD) reduces the amount of circulating IL-1β in plasma samples obtained at 12 months after PFF-Synuclein injections in both a prophylactic and therapeutic setting (n=4-6 for each group). *c.f Saline, #c.f PFF-Synuclein, ns=not significant. Data are Mean±SEM ***###P<0.001****####P<0.0001, by one-way analysis of variance (ANOVA), and Tukey's post test.

FIG. 12: Study H—Oral treatment with the compound of formula (I) (CPD) protects against nigrostriatal dopaminergic degeneration in the PFF-Synuclein model of Parkinson's disease. A) Dopamine on the ipsilateral striatum of PFF mice at 12 months (n=4-8 per group) B) DOPAC on the ipsilateral striatum of PFF mice at 12 months (n=4-8 per group) C) HVA on the ipsilateral striatum of PFF mice at 12 months (n=4-8 per group). *c.f Saline, *c.f PFF-Synuclein, Data are mean±SEM. ns=not significant. *#P<o.o5,**##P<0.01, ****####P<0.0001, by one-way analysis of variance (ANOVA), and Tukey's post test.

### STUDY A—BLOOD-BRAIN BARRIER PENETRATION IN HEALTHY MICE

Objective

The present study was designed to determine the free concentration of the compound of formula (I) in the left and right striatum of freely-moving adult male mice after oral administration.

Animals

Adult male C57B1/6 mice (22-28 g; Envigo, the Netherlands) were used for the experiments. Following arrival, animals were housed in groups of 5 in polypropylene cages (40×50×20 cm) with wire mesh top in a temperature (22±2° C.) and humidity (55±15%) controlled environment on a 12 hour light cycle (07.00-19.00). Following surgery, animals were housed individually (cages 30×30×40 cm). Standard diet (SDS Diets, RM1 PL) and domestic quality mains water were available ad libitum.

Surgery

Mice were anesthetized using isoflurane (2% and 500 mL/min O₂). Before surgery, Finadyne (1 mg/kg, s.c.) was administered for analgesia during surgery and the post-surgical recovery period. A mixture of bupivacaine and epinephrine was used for local analgesia of the incision site.

Microdialysis Probe Implantation

The animals were placed in a stereotaxic frame (Kopf instruments, USA). MetaQuant microdialysis probes with a 3 mm exposed polyacrylonitrile membrane (MQ-PAN 33) were implanted bilaterally into the left and right striatum (coordinates for the tip of the probe: AP=+0.8 mm (to bregma), ML=+/−1.7 mm (to midline), DV=−4.0 mm (to dura) with an angle of 0° and the incisor bar set at 0.0 mm. All coordinates were based on “The mouse brain in stereotaxic coordinates” by Paxinos and Franklin (2008). The probes were attached to the skull with a stainless-steel screw and dental cement.

Dose Formulations

The monosodium salt of the compound of formula (I) was formulated in sterilized tap water at concentrations (with respect to the non-salt form) of 0.2 and 4 mg/mL for oral dosing at 5 mL/kg; 1 mg/kg and 20 mg/kg, respectively. The dose formulations are shown in Table 1. The administered volumes for each animal are shown in Table 2.

TABLE 1
Dose formulations

| Formulation | Monosodium salt amount | Solvent |
|---|---|---|
| A | 1.31 mg | 6.19 mL sterilised tap water |
| B | 1.81 mg | 0.428 mL sterilised tap water |
| C | 2.39 mg | 0.565 mL sterilised tap water |

TABLE 2
Compound administrations

| | | | |
|---|---|---|---|
| | Weight | | Volume |
| Mouse ID | (g) | Formulation | administered (mL) |

1 mg/kg

| | | | |
|---|---|---|---|
| 2015341-364-3530 | 22 | A | 0.11 |
| 2015341-362-3532 | 28 | A | 0.14 |
| 2015341-363-3531 | 22 | A | 0.11 |
| 2015341-366-3528 | 28 | A | 0.14 |

20 mg/kg

| | | | |
|---|---|---|---|
| 2015341-365-3529 | 25 | B | 0.13 |
| 2015341-367-3495 | 25 | B | 0.13 |
| 2015341-379-3488 | 27 | C | 0.14 |
| 2015341-378-3489 | 25 | C | 0.12 |

Experimental Design

The MetaQuant microdialysis probes were connected with flexible PEEK tubing (Western Analytical Products Inc. USA; PK005-020) to a microperfusion pump (Harvard) and perfused with a slow flow of artificial CSF (perfusate), containing 147 mM NaCl, 3.0 mM KCl, 1.2 mM CaCl₂, and 1.2 mM MgCl₂, at a flow rate of 0.12 μL/min and a carrier flow of UP+0.02 M FA+0.04% ascorbic acid at o.8 μL/min. After a minimum of two hours of prestabilisation, microdialysis samples were collected in 60 minute intervals. Following collection of two baseline samples, the compound of formula (I) (1 or 20 mg/kg in sterilised tap water) was administered orally at t=0 minutes. The specific microdialysis sampling schedule is shown in Table 3. Samples were collected into mini-vials (Microbiotech/se AB, Sweden; 4001029) using an automated fraction collector (UV 8301501, TSE, Univentor, Malta). At the end of the experiment, the animals were sacrificed.

TABLE 3
Microdialysis sampling schedule

| Sample | Time | |
|---|---|---|
| number | (min) | Action |
| A1 | | Flow check (30 min sample) - no analysis |
| A2 | | Flow check (30 min sample) - no analysis |
| 101 | −60 | Basal sample |
| 102 | 0 | Basal sample; administer compound after this |
| | | sample has been taken, at t = 0 min |
| 103 | 60 | |
| 104 | 120 | |
| 105 | 180 | |
| 106 | 240 | |
| 107 | 300 | |
| 108 | 360 | |
| 109 | 420 | |
| 110 | 480 | Turn slow flow off after this sample, wait 15 min |
| A3 | 525 | Flow check (30 min sample) - no analysis |
| A4 | 555 | Flow check (30 min sample) - no analysis |

Bioanalysis

Microdialysate samples from MetaQuant probes contained a nominal volume of 55.2 μL dialysate. Levels of the compound of formula (I) in MetaQuant microdialysate samples were quantified by LC-MS/MS.

The dialysate samples were mixed with acetonitrile and an aliquot of this mixture was injected into the LC system by an automated sample injector (SIL-20AD, Shimadzu, Japan). Calibrators and in-run QC samples were prepared in analytical dialysate of the same composition as the microdialysate samples.

Chromatographic separation of the compound was performed on a reversed phase column (100×3.0 mm, particle size 2.5 μm, Phenomenex) held at a temperature of 40° C. in a gradient elution run, using eluent B (acetonitrile+0.1% formic acid) in eluent A (ultrapurified water+0.1% formic acid) at a flow rate of 0.3 mL/min.

MS analyses were performed using an API 4000 MS/MS system consisting of an API 4000 MS/MS detector and a Turbo Ion Spray interface (both from Applied Biosystems, USA). The acquisitions were performed in positive ionization mode, with ionization spray voltage set at 5.5 kV. The probe temperature was set at 550° C. The instrument was operated in multiple-reaction-monitoring (MRM) mode.

MRM transitions for the analyte are shown in Table 4. Suitable in-run calibration curves were fitted using weighted (1/x) regression and the sample concentrations were determined using these calibration curves. Accuracy was verified by quality control samples after each sample series. Concentrations were calculated with the Analyst™ data system (Applied Biosystems).

TABLE 4
MRM table

| Analyte | Q1 | Q3 |
|---|---|---|
| Compound of formula (I) | 387 | 190 |

Data Evaluation

Pharmacokinetic data for the compound of formula (I) is presented as concentrations (mean+SEM) in microdialysate, corrected for dilution during the experiment. Pharmacokinetic data for the compound of formula (I) in microdialysate was not corrected for recovery. Results were plotted in Prism 5 for Windows (GraphPad Software).

Results

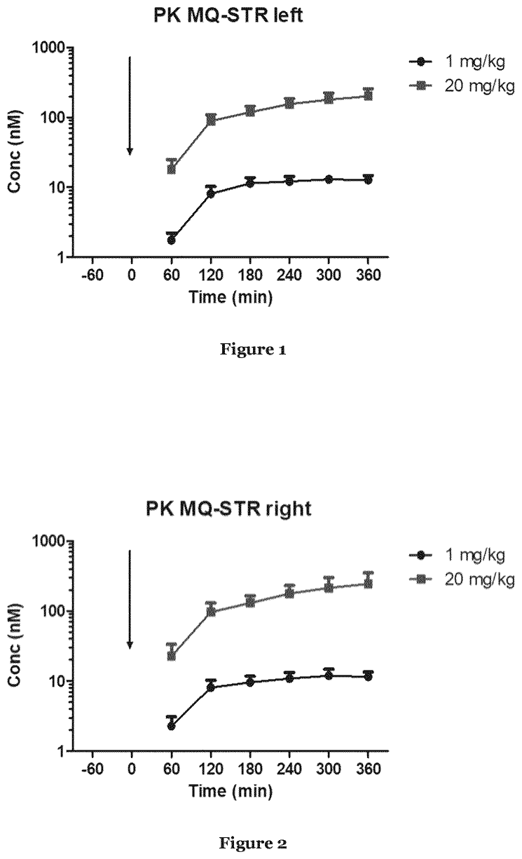

FIG. 1 shows the absolute levels of the compound of formula (I) in the MetaQuant dialysate from the left striatum of freely-moving adult male C57B^{1/6} mice following oral administration of 1 or 20 mg/kg of the compound. FIG. 2 shows the absolute levels of the compound of formula (I) in the MetaQuant dialysate from the right striatum of freely-moving adult male C57B^{1/6} mice following oral administration of 1 or 20 mg/kg io of the compound. 1 mg/kg dosed animals showed average peak levels of 12-13 nM in both the left and right striatal dialysate samples at 5 hours after compound administration. 20 mg/kg dosed animals showed average peak levels of 201-243 nM in both the left and right striatal dialysate samples at 6 hours after compound administration.

As is evident, the results demonstrate the ability of the compound of formula (I) to cross the blood-brain barrier following oral administration. The compound of formula (I) has previously been demonstrated to be a highly effective inhibitor of the activation of the NLRP3 inflammasome (see WO 2016/131098, which is incorporated in its entirety herein by reference). Moreover, inhibition of the NLRP3 inflammasome has been implicated in the treatment of disorders such as Parkinson's disease, Alzheimer's disease, Motor Neurone disease (Amyotrophic Lateral Sclerosis), Huntington's disease, Multiple System Atrophy, Progressive Supranuclear Palsy, Frontotemporal Dementia, Ataxia, and Neurodegenerative Prion Diseases (see Walsh et al., Nature Reviews, 15: 84-97, 2014; Dempsey et al., Brain Behav Immun, 61: 306-316, 2017; Fangzhou et al., J Neuropathol Exp Neurol, 77(11): 1055-1065, 2018; Ising et al., Nature, 575: 669-673, 2019; Kojic et al., Nature Communications, 9: 395, 2018; and Shi et al., J Neuroinflamm, 9: 73, 2012, all of which are incorporated in their entirety herein by reference). As such, it is believed that the compound of formula (I) will be effective in the treatment or prevention of neurodegenerative disorders.

Study B—Blood-Brain Barrier Penetration in the 6-OHDA Mouse Model of Parkinson's Disease

Objective

The present study was designed to assess the free concentration of the compound of formula (I) in the left and right striatum of freely-moving adult male mice with a unilateral 6-hydroxydopamine (6-OHDA) lesion.

Animals

Adult male C57B1/6 mice (23-28 g; Envigo, the Netherlands) were used for the experiments. Following arrival, animals were housed in groups of 5 in polypropylene cages (40×50×20 cm) with wire mesh top in a temperature (22±2° C.) and humidity (55±15%) controlled environment on a 12 hour light cycle (07.00-19.00). Following surgery, animals were housed individually (cages 30×30×40 cm). Standard diet (SDS Diets, RM1 PL) and domestic quality mains water were available ad libitum.

Surgery

Mice were anesthetized using isoflurane (2% and 500 mL/min O₂). Before surgery, Finadyne (1 mg/kg, s.c.) was administered for analgesia during surgery and the post-surgical recovery period. A mixture of bupivacaine and epinephrine was used for local analgesia of the incision site.

6-OHDA Lesion

The animals were placed in a stereotaxic frame (Kopf instruments, USA). 10 μg of 6-OHDA in 2 μL saline was slowly injected into the right striatum using a Hamilton needle (coordinates for the tip of the needle: AP=−0.5 mm (to bregma), ML=−2.0 mm (to midline), DV=-4.0 mm (to dura) with an angle of 0° and the incisor bar set at 0.0 mm.

Microdialysis Probe Implantation

In the same surgical procedure, guides for MetaQuant microdialysis probes with a 3 mm exposed polyacrylonitrile membrane (MQ-PAN 3/3) were implanted bilaterally into the left and right striatum (coordinates for the tip of the probe: AP=+0.8 mm (to bregma), ML=+/−1.7 mm (to midline), DV=−4.0 mm (to dura) with an angle of 0° and the incisor bar set at 0.0 mm. All coordinates were based on “The mouse brain in stereotaxic coordinates” by Paxinos and Franklin (2008). The probes were attached to the skull with a stainless-steel screw and dental cement.

Dose Formulations

The monosodium salt of the compound of formula (I) was formulated in sterilized tap water at concentrations of 0.2 and 4 mg/mL for oral dosing at 5 mL/kg; 1 mg/kg and 20 mg/kg, respectively. The dose formulations are shown in Table 5. The administered volumes for each animal are shown in Table 6.

TABLE 5
Dose formulations

| Formulation | Monosodium salt amount | Solvent |
|---|---|---|
| D | 1.32 mg | 6.24 mL sterilised tap water |
| E | 3.89 mg | 0.919 mL sterilised tap water |
| F | 1.25 mg | 5.91 mL sterilised tap water |

TABLE 6
Compound administrations

| | | | |
|---|---|---|---|
| | Weight | | Volume |
| Mouse ID | (g) | Formulation | administered (mL) |

1 mg/kg

| | | | |
|---|---|---|---|
| 2015341-390-3487 | 28 | D | 0.14 |
| 2015341-391-3486 | 26 | D | 0.13 |
| 2015341-392-3485 | 23 | D | 0.12 |
| 2015341-413-3474 | 28 | F | 0.14 |

20 mg/kg

| | | | |
|---|---|---|---|
| 2015341-394-3483 | 24 | E | 0.12 |
| 2015341-395-3482 | 24 | E | 0.12 |
| 2015341-396-3481 | 24 | E | 0.12 |
| 2015341-397-3480 | 25 | E | 0.12 |

Experimental Design

After recovery from lesion and the guide surgery, on day 10, MetaQuant microdialysis probes were connected with flexible PEEK tubing (Western Analytical Products Inc. USA; PK005-020) to a microperfusion pump (Harvard) and perfused with a slow flow of artificial CSF (perfusate), containing 147 mM NaCl, 3.0 mM KCl, 1.2 mM CaCl₂, and 1.2 mM MgCl₂, at a flow rate of 0.12 μL/min and a carrier flow of UP+0.02 M FA+0.04% ascorbic acid at 0.8 μL/min. After a minimum of two hours of prestabilisation, microdialysis samples were collected in 60 minute intervals. Following collection of two baseline samples, the compound of formula (I) (1 or 20 mg/kg in sterilised tap water) was administered orally at t=0 minutes. The specific microdialysis sampling schedule is shown in Table 7. Samples were collected into mini-vials (Microbiotech/se AB, Sweden; 4001029) using an automated fraction collector (UV 8301501, TSE, Univentor, Malta). At the end of the experiment, the animals were sacrificed.

TABLE 7
Microdialysis sampling schedule

| Sample | Time | |
|---|---|---|
| number | (min) | Action |
| A1 | | Flow check (30 min sample) - no analysis |
| A2 | | Flow check (30 min sample) - no analysis |
| 101 | −60 | Basal sample |
| 102 | 0 | Basal sample; administer compound after this |
| | | sample has been collected, at t = 0 min |
| 103 | 60 | |
| 104 | 120 | |
| 105 | 180 | |
| 106 | 240 | |
| 107 | 300 | |
| 108 | 360 | |
| 109 | 420 | |
| 110 | 480 | Turn slow flow off after this sample, wait 15 min |
| A3 | 525 | Flow check (30 min sample) - no analysis |
| A4 | 555 | Flow check (30 min sample) - no analysis |

Bioanalysis Microdialysate samples from MetaQuant probes contained a nominal volume of 55.2 μL dialysate and were used without further sample preparation.

Levels of the compound of formula (I) in MetaQuant microdialysate samples were quantified by LC-MS/MS. An aliquot of the dialysate sample was mixed with acetonitrile and of this mixture an aliquot was injected into the LC system by an automated sample injector (SIL-20AD, Shimadzu, Japan). Calibrators and in-run QC samples were prepared in analytical dialysate of the same composition as the microdialysate samples.

Chromatographic separation of the compound was performed on a reversed phase column (100×3.0 mm, particle size 2.5 μm, Phenomenex) held at a temperature of 40° C. in a gradient elution run, using eluent B (acetonitrile+0.1% formic acid) in eluent A (ultrapurified water+0.1% formic acid) at a flow rate of 0.3 mL/min.

MS analyses were performed using an API 4000 MS/MS system consisting of an API 4000 MS/MS detector and a Turbo Ion Spray interface (both from Applied Biosystems, USA). The acquisitions were performed in positive ionization mode, with ionization spray voltage set at 5.5 kV. The probe temperature was set at 550° C. The instrument was operated in multiple-reaction-monitoring (MRM) mode.

MRM transitions for the analyte are shown in Table 8. Suitable in-run calibration curves were fitted using weighted (1/x) regression and the sample concentrations were determined using these calibration curves. Accuracy was verified by quality control samples after each sample series. Concentrations were calculated with the Analyst™ data system (Applied Biosystems).

TABLE 8
MRM table for the compound of formula (I)

| Analyte | Q1 | Q3 |
|---|---|---|
| Compound of formula (I) | 387 | 190 |

Data Evaluation

Pharmacokinetic data for the compound of formula (I) are presented as concentrations (mean+SEM) in microdialysate, corrected for dilution during the experiment. Pharmacokinetic data for the compound were not corrected for recovery (recovery of the compound of formula (I) is 61% as per BOL key 1344). Results were plotted in Prism 5 for Windows (GraphPad Software).

Results

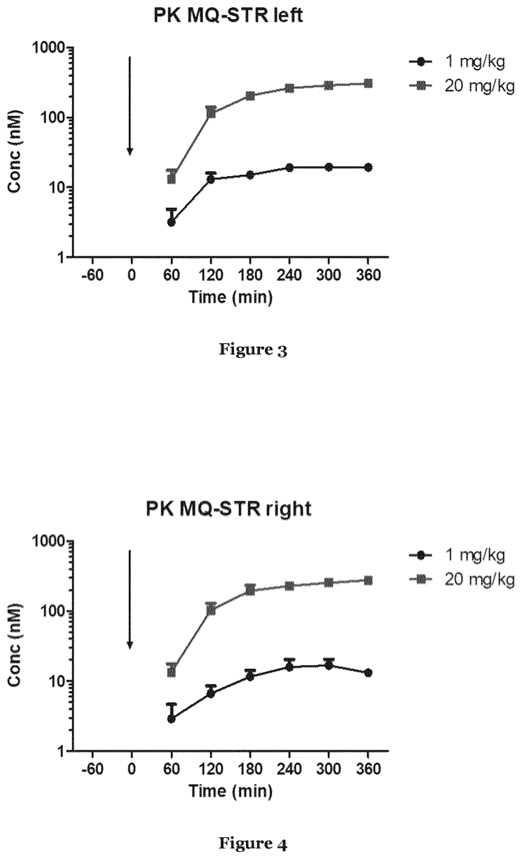

FIG. 3 shows the absolute levels of the compound of formula (I) in the MetaQuant dialysate from the left striatum of freely-moving adult male C57B^{1/6} mice following oral administration of 1 or 20 mg/kg of the compound. FIG. 4 shows the absolute levels of the compound of formula (I) in the MetaQuant dialysate from the right striatum of freely-moving adult male C57B^{1/6} mice following oral administration of 1 or 20 mg/kg of the compound.

1 mg/kg dosed animals showed average peak levels of 17-19 nM of the compound of io formula (I) in both the left and right striatal dialysate samples at 5 hours after compound administration. 20 mg/kg dosed animals showed average peak levels of 280-300 nM of the compound of formula (I) in both the left and right striatal dialysate samples at 6 hours after compound administration.

Thus, it can be seen that the ability of the compound of formula (I) to cross the blood-brain barrier following oral administration is similar in both healthy mice and mice suffering from an animal model of Parkinson's disease.

Study C—Oral Efficacy in the 6-OHDA Mouse Model of Parkinson's Disease

Objective

To determine the oral efficacy of the compound of formula (I) in the 6-OHDA mouse model of Parkinson's disease.

Treatment

8-week-old C57BL6 male mice (obtained from ARC, Perth, Australia) were housed under a 12-h light cycle in a SPF climate-controlled facility with food and water provided ad libitum for two weeks prior to study initiation. For treatment with the compound of formula (I), mice were dosed via oral gavage. Ten (10) mice in each group were dosed at 3 or 1 mg/kg, starting the day before (24 hr prior) stereotaxic surgery, and then QD until sacrifice.

6-OHDA Preparation

6-OHDA (Sigma) was prepared immediately prior to surgeries. A sterile saline (0.9%) solution containing ascorbic acid (0.2%) was used as the vehicle to dissolve 6-OHDA. Ascorbic acid was used to stabilize 6-OHDA, as it prevents its oxidation to an inactive form. In order to inject a final concentration of 12 μg into the right striatum, a working stock of 6 mg/ml was made injecting a final volume of 2 μl.

Surgical Procedure

Mice were anaesthetized using ketamine (100 mg/kg, i.p.) and xylazine (10 mg/kg i.p.)

anesthesia and were placed into a stereotactic frame with nose and ear bars specially adapted for mice. The lesion was performed using a 5 μl Hamilton syringe to deliver either vehicle or 6-OHDA (12 μg) at the following coordinates relative to bregma: AP: −1.2 mm; ML: −1.7 mm; DV: 3.5 mm into the right dorsal striatum according to the stereotaxic atlas (“The mouse brain in stereotaxic coordinates” by Paxinos and Franklin, 1997). After drilling a 1 mm burr-hole in the skull, a 2 μl volume of solution was infused at the target site at the rate of 0.5 μl per minute. The needle was held in place for at least 5 minutes after injection to minimize retrograde flow along the needle tract. Mice were administered a subcutaneous injection of sterile Ringer's solution to is facilitate recovery and were placed on a heat-pad until complete recovery from anesthesia.

Amphetamine Induced Rotations

Amphetamine-induced ipsilateral rotations were performed at day 21 post-surgery. Mice were injected with 2 mg/kg of D-amphetamine and placed in circular glass bowls.

After an acclimatization period of five minutes, the net ipsilateral rotations over ten minutes were recorded and counted. Quantitation was performed from recorded videos by an investigator blinded to the treatment groups.

LC-MS/MS Quantification of Striatal Dopamine and Metabolites

Mice were sacrificed 1 week after the amphetamine test (day 28) and striatal tissue was micro-dissected, weighed and snap-frozen at −80° C. Neurotransmitters from striatal tissues were extracted and derivatized using ethyl chloroformate. Striatal dopamine (DA) and its metabolites (DOPAC and HVA) were quantified in their stable derivative 3o form in the presence of internal standard 3,4-dihydroxybenzylamine (DHBA) using highly sensitive liquid chromatography-tandem mass spectrometry (LC-MS/MS) as described previously (Park et al., Biol. Pharm. Bull., 2013, vol. 36, pp. 252-8). An API 3200 (AB SCIEX) triple quadrupole Q TRAP LC/MS/MS system was used with Turbo V ion source coupled with Agilent series HPLC system under positive (+1) ionization in multi-reaction mode. Samples were chromatographed on a Phenomenex synergi fusion −RP 80 Å analytical column (150×4.6 mm; 4 μm) under a binary gradient condition at 500 μl flow rate using mobile phase A (0.1% formic acid in milliQ water) and mobile phase B (0.1% formic acid in acetonitrile). For quantitation, one transition per analyte was monitored and two transitions per analyte were monitored for qualitative purposes.

Results

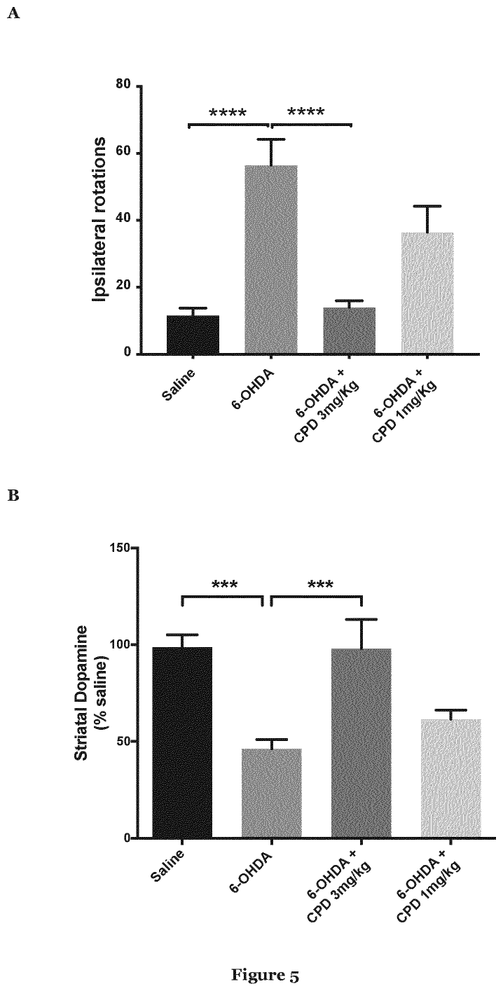

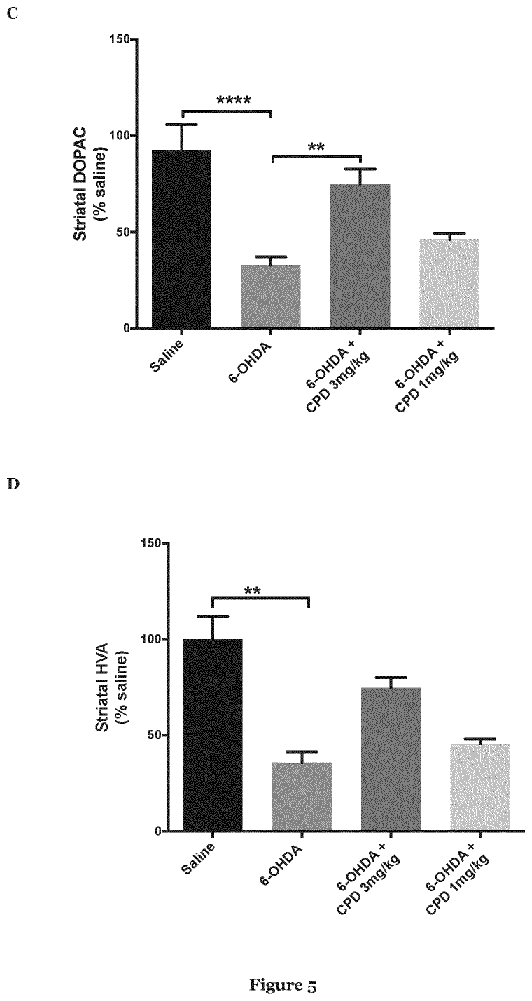

In this study the efficacy of the compound of formula (I) at 3 and 1 mg/kg was compared. The results are shown in FIG. 5. At 3 mg/kg, a significant protective effect from amphetamine-induced ipsilateral rotations was found. The quantification of striatal dopamine and its metabolites DOPAC and HVA at 28 days after the 6-OHDA lesion further confirmed that mice treated with 3 mg/kg of the compound of formula (I) were significantly protected against the loss of dopamine. Accordingly, it can be concluded that daily oral administration of the compound of formula (I) protects against nigrostriatal dopaminergic degeneration in experimental Parkinson's disease.

Study D—Comparison with MCC95o in the 6-OHDA Mouse Model of Parkinson's Disease

Objective and Procedure

MCC950 is a previously reported NLRP3 inhibitor (see Coll et al., Nature Medicine, 2015, vol. 21(3), pp. 248-255, which is incorporated in its entirety herein by reference) having the following formula:

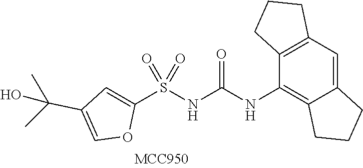 [SMILES: CC(C)(O)c1coc(S(=O)(=O)NC(=O)Nc2c3c(cc4c2CCC4)CCC3)c1]

The aim of study D was to compare the neuroprotective efficacy of the compound of formula (I) with MCC95o in the mouse unilateral 6-OHDA model at 3 mg/kg. The amphetamine-induced ipsilateral rotations and striatal dopamine (DA) levels were assessed, using a protocol identical to that used for study C, with ten (10) mice in each group being dosed at 3 mg/kg for each drug, starting the day before (24 hr prior) stereotaxic surgery, and then daily throughout until sacrifice.

Results

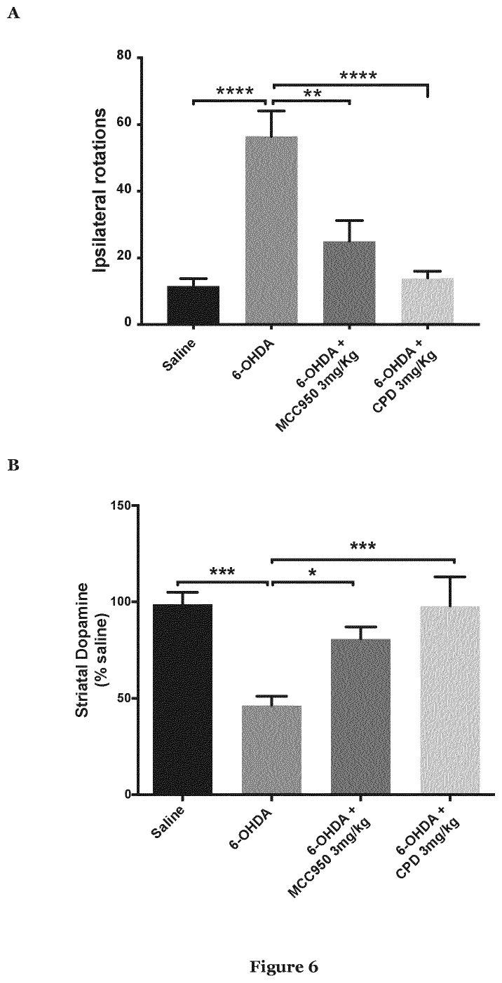

The results are shown in FIG. 6. At 3 mg/kg, a significant protective effect from amphetamine-induced ipsilateral rotations was found for both drugs. However, the mice treated with the compound of formula (I) had fewer ipsilateral rotations compared to MCC950 (FIG. 6A). Specifically, a 75% reduction in amphetamine-induced rotations was observed for the compound of formula (I) compared to a 55% reduction for MCC950 relative to the 6-OHDA untreated group. These behavioural results are also reflected in the amount of striatal dopamine. The quantification of striatal dopamine (FIG. 6B) at 28 days after the 6-OHDA lesion further confirmed io that mice treated with 3mg/kg of the compound of formula (I) were significantly protected against the loss of dopamine with improved efficacy compared to MCC950. It can therefore be seen that the compound of formula (I) has improved efficacy over MCC950 in the 6-OHDA mouse model of Parkinson's disease.

Study E—Comparison with MCC950 in Inhibiting NLRP3 Inflammasome in Primary Microglia

Objective

To determine the IC₅₀ of the compound of formula (I) and MCC950 in LPS primed microglia activated with the canonical NLRP3 activator ATP.

Primary Microglia Cultures

Primary microglial cultures were prepared from C57BL/6 postnatal day 1 (P1) mouse pups and purified by column free magnetic separation system as described previously (see Gordon et al., J. Neurosci. Methods, 2011, vol. 194(2), pp. 287-296, which is incorporated in its entirety herein by reference). Primary microglia were maintained in DMEM/F12 complete medium (DMEM-F12, GIBCO supplemented with 10% heat-inactivated FBS, 50 U/mL penicillin, 50 μg/mL streptomycin, 2 mM L-glutamine, 100 μM nonessential amino acids, and 2 mM sodium pyruvate). Cells were then maintained in a 5% CO₂ incubator at 37° C.

IL-1β ELISA for IC₅₀ Determination

The mouse IL-1β kit (R&D Systems, Catalog #DY008), was used to measure IL-1β level in the supernatants of LPS primed microglia (3 hours 200 ng/ml) pre-treated with increasing concentrations of MCC950 and the compound of formula (I), and activated with ATP 5 mM for 1 hour.

Results

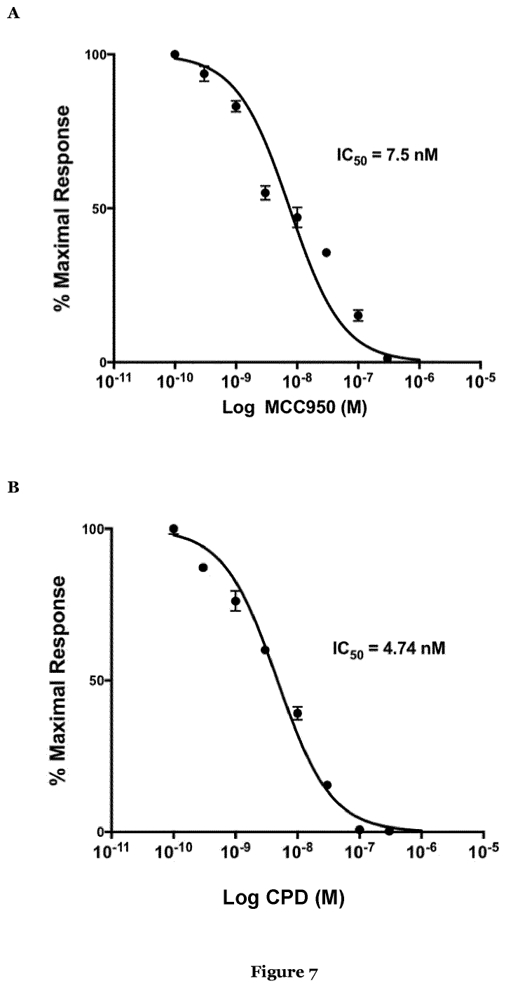

The results are shown in FIG. 7. MCC95o obtained an IC₅₀ of 7.5 nM (FIG. 7A), whereas the compound of formula (I) displayed a potency of 4.7 nM under the same conditions (FIG. 7B). Thus, the compound of formula (I) displays increased potency compared with MCC950 in inhibiting NLRP3 inflammasome in primary microglia.

Study F—Inhibition of the NLRP3 Inflammasome in Primary Human Microglia from a Healthy Brain

Objective

To determine the IC₅₀ of the compound of formula (I) in LPS primed human microglia activated with the canonical NLRP3 activator ATP.

Human Brain Samples

Human brain material was obtained via the rapid autopsy system of the Netherlands Brain Bank (NBB; Amsterdam, the Netherlands), which supplies post mortem material from clinically well-documented and neuropathological confirmed cases and non-neurological controls. Autopsies were performed on donors from whom written informed consent had been obtained by the NBB. One (1) healthy brain tissue sample was used in this experiment.

Microglia Isolation Method

Human adult microglia cells were isolated and cultured as previously described by Bsibsi et al. (Journal of Neuropathology & Experimental Neurology, 2002, vol. 61(11), pp. 1013-1021). Briefly, at the Netherlands Brain Bank (Amsterdam, The Netherlands), tissue samples were dissected from subcortical white matter and stored in tubes with culture medium at 4° C. The samples were then transported to the laboratory of Charles River Laboratories (Leiden, The Netherlands) in tubes with culture medium. Visible blood vessels were removed and brain tissue was washed with PBS. After a 20-min digestion in 0.25% trypsin the cell suspension was gently triturated and washed with DMEM/HAM-F12 medium containing 10% FCS and antibiotic supplements. After passage through a 100-μm filter, myelin was removed by Percoll gradient centrifugation. Erythrocytes were lysed by 15-min incubation on ice with 155 mM NH₄Cl, 1 mM KHCO₃ and 0.2% BSA in PBS. Next, the cell suspension was seeded into non-coated 96-well plates at a density of 40000-100000 cells/well. To promote proliferation and survival of microglial cells, recombinant human GM-CSF was added to the culture medium at seeding and every 3 days thereafter at a final concentration of 20 ng/ml. After 3-5 days, cultures were washed with medium to remove debris; this was defined as day o for the assay. The purity of the cultured microglial cells was verified by immunostaining for microglial identity marker (Iba1) and activation marker (CD45). In addition, cultures were checked for potential contaminating cell populations including astrocytes (GFAP expression) and neurons (NeuN expression). The QC plates were fixed with 4% formaldehyde on the same day of the experiment start.

IL-1β ELISA for IC₅₀ Determination

At day o myelin and cell debris was removed by washing with medium. At day 2 and 3 (T=0 h), culture medium was replaced with 80 μl 100 ng/ml LPS (prepared in serum free medium) to prime microglia. At T=+1.5 h 1000 nM, 200 nM, 40 nM, 8 nM, 1.6 nM, 0.3 nM, 0.064 nM of the compound of formula (I) (in PBS) was added. After 30 min, 5 mM ATP (final concentration, in serum free media) was added to the cultures. At different time points post trigger, supernatants were collected in separate 96-well plates and stored at −20° C. (samples analysed were collected at 2 hour post ATP addition). A Meso Scale Discovery (MSD®) cytokine immunoassay (U-PLEX Human Kit) was used to quantify concentrations of IL-1β in the cell supernatants from each condition, according to manufacturer's instructions provided with the kit (MSD #K151TUK-2). Briefly, MSD plates were coated with capture antibody diluted in Diluent 100 at room temperature for 2 hours on a shaker platform. Plates were washed with o.o5% PBS-Tween, and 25 μL per well of diluent 43 and 25 μL per well of the undiluted samples and standard curve concentrations in technical duplicates were added and incubated overnight at 4° C. while shaking (500 rpm). Plates were washed with 0.05% PBS-Tween, and MSD Sulfo-Tag-conjugated detection antibody diluted in diluent 3 was added to each well and incubated for 1 hour at room temperature while shaking. Plates were then washed with o.o5% PBS-Tween, and 150 μl of MSD Read Buffer-T 4× (with surfactant) diluted 1:2 in water was added to each well. The plates were read using an MSD sector imager model 6000 and the concentration was calculated using MSD discovery workbench® version 4. Samples were analyzed on an MSD SECTOR S 600 reader and DISCOVERY WORKBENCH analyzed complex set of data generated from MSD plates.

Results

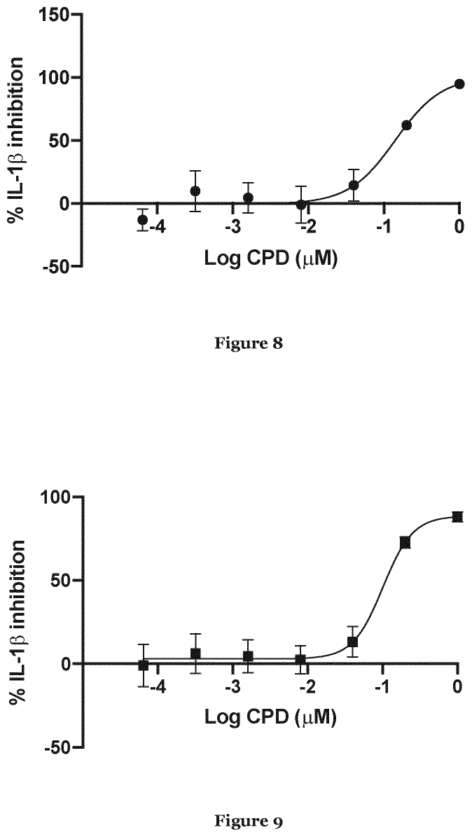

IL-1β concentrations in the supernatants were back-calculated using standard curves of recombinant IL-1β included in the MSD kits. As shown in FIG. 8 the compound of formula (I) obtained an IC₅₀ of 142 nM, thus demonstrating that the compound is effective at inhibiting IL-1β production in human microglia.

Study G—Inhibition of the NLRP3 Inflammasome in Primary Human Microglia from Parkinson's Brains

Objective

To determine in a disease context the IC₅₀ of the compound of formula (I) in LPS primed human microglia activated with the canonical NLRP3 activator ATP.

Human Brain Samples

Human brain material was obtained via the rapid autopsy system of the Netherlands Brain Bank (NBB; Amsterdam, the Netherlands), which supplies post mortem material from clinically well-documented and neuropathological confirmed cases and non-neurological controls. Autopsies were performed on donors from whom written informed consent had been obtained by the NBB. Three Parkinson's brains were used in these experiments.

Microglia Isolation Method

Human adult microglia cells were isolated and cultured as previously described by Bsibsi et al. (Journal of Neuropathology & Experimental Neurology, 2002, vol. 61(11), pp. 1013-1021). Briefly, at the Netherlands Brain Bank (Amsterdam, The Netherlands), tissue samples were dissected from subcortical white matter and stored in tubes with culture medium at 4° C. The samples were then transported to the laboratory of Charles River Laboratories (Leiden, The Netherlands) in tubes with culture medium. Visible blood vessels were removed and brain tissue was washed with PBS. After a 20-min digestion in 0.25% trypsin the cell suspension was gently triturated and washed with DMEM/HAM-F12 medium containing 10% FCS and antibiotic supplements. After passage through a 100-μm filter, myelin was removed by Percoll gradient centrifugation. Erythrocytes were lysed by 15-min incubation on ice with 155 mM NH₄Cl, 1 mM KHCO₃ and 0.2% BSA in PBS. Next, the cell suspension was seeded into non-coated 96-well plates at a density of 40000-100000 cells/well. To promote proliferation and survival of microglial cells, recombinant human GM-CSF was added to the culture medium at seeding and every 3 days thereafter at a final concentration of 20 ng/ml. After 3-5 days, cultures were washed with medium to remove debris; this was defined as day o for the assay. The purity of the cultured microglial cells was verified by immunostaining for microglial identity marker (Iba1) and activation marker (CD45). In addition, cultures were checked for potential contaminating cell populations including astrocytes (GFAP expression) and neurons (NeuN expression). The QC plates were fixed with 4% formaldehyde on the same day of the experiment start.

IL-1β ELISA for IC₅₀ Determination

At day o myelin and cell debris was removed by washing with medium. At day 2 and 3 (T=0 h), culture medium was replaced with 80 μl wo ng/ml LPS (prepared in serum free medium) to prime microglia. At T=+1.5 h 1000 nM, 200 nM, 40 nM, 8 nM, 1.6 nM, 0.3 nM, 0.064 nM of the compound of formula (I) (in PBS) was added. After 30 min, 5 mM ATP (final concentration, in serum free media) was added to the cultures. At different time points post trigger, supernatants were collected in separate 96-well plates and stored at −20° C. (samples analysed were collected at 2 hour post ATP addition). A Meso Scale Discovery (MSD®) cytokine immunoassay (U-PLEX Human Kit) was used to quantify concentrations of IL-1β in the cell supernatants from each condition, according to manufacturer's instructions provided with the kit (MSD #K151TUK-2). Briefly, MSD plates were coated with capture antibody diluted in Diluent 100 at room temperature for 2 hour on a shaker platform. Plates were washed with 0.05% PBS-Tween, and 25 μL per well of diluent 43 and 25 μL per well of the undiluted samples and standard curve concentrations in technical duplicates were added and incubated overnight at 4° C. while shaking (soo rpm). Plates were washed with 0.05% PBS-Tween, and MSD Sulfo-Tag-conjugated detection antibody diluted in diluent 3 was added to each well and incubated for 1 hour at room temperature while shaking. Plates were then washed with 0.05% PBS-Tween, and 150 μl of MSD Read Buffer-T 4× (with surfactant) diluted 1:2 in water was added to each well. The plates were read using an MSD sector imager model 6000 and the concentration was calculated using MSD discovery workbench® version 4. Samples were analyzed on an MSD SECTOR S 600 reader and DISCOVERY WORKBENCH analyzed complex set of data generated from MSD plates.

Results

IL-1β concentrations in the supernatants were back-calculated using standard curves of recombinant IL-1β included in the MSD kits. As shown in FIG. 9 the compound of formula (I) obtained an IC₅₀ of 101 nM, thus demonstrating that the compound is effective at inhibiting IL-1β production in human microglia in a disease context.

Microglia are located in the brain and spinal cord, and act as the main form of active immune defence in the central nervous system. The inflammatory response in microglia is implicated in disorders such as Parkinson's disease (see Ho, Adv. Exp. Med. Biol., 2019, vol. 1175, pp. 335-353; and Gordon et al., Sci. Transl. Med., 2018, vol. 10(465), which are incorporated in their entirety herein by reference), Alzheimer's disease (see Hemonnot et al., Front. Aging Neurosci., 2019, vol. 11, article 233, which is incorporated in its entirety herein by reference), Motor Neurone disease (Amyotrophic Lateral Sclerosis) (see Rodriguez et al., Current Medicinal Chemistry, 2016, vol. 23(42), pp. 4753-4772; and Brites et al., Front. Cell. Neurosci., 2014, vol. 8, article 117, which are incorporated in their entirety herein by reference), Huntington's disease (see Yang et al., Front. Aging Neurosci., 2017, vol. 9, article 193; and Pavese et al., Neurology, 2006, vol. 66(11), pp. 1638-1643, which are incorporated in their entirety herein by reference), Multiple System Atrophy (see Kübler et al., Mov. Disord., 2019, vol. 34(4), pp. 564-568; and Ishizawa et al., J. Neuropath. Exp. Neurol., 2004, vol. 63(1), pp. 43-52, which are incorporated in their entirety herein by reference), Progressive Supranuclear Palsy (see Fernández-Botrán et al., Parkinsonism Relat Disord., 2011, vol. 17(9), pp. 683-688; and Ishizawa et al., J. Neuropath. Exp. Neurol., 2001, vol. 60(6), pp. 647-57, which are incorporated in their entirety herein by reference), Frontotemporal Dementia (see Pasqualetti et al., Current Neurology and Neuroscience Reports, 2015, vol. 15, article 17; Bachiller et al., Front. Cell. Neurosci., 2018, vol. 12, article 488; and Cagnin et al., Ann. Neurol., 2004, vol. 56(6), pp. 894-7, which are incorporated in their entirety herein by reference), Ataxia (see Kojic et al., Nature Communications, 9: 3195, 2018, which is incorporated in its entirety herein by reference), and Neurodegenerative Prion Diseases (see Shi et al., J Neuroinflamm, 9: 73, 2012, which is incorporated in its entirety herein by reference). The results presented herein demonstrate both (i) that the compound of formula (I) is a highly potent inhibitor of NLRP3 in microglia, and (ii) that it is able to reach such microglia by crossing the blood-brain barrier following oral administration. As such, it is believed that the compound of formula (I) will be effective in the treatment or prevention of neurodegenerative disorders.

Study H—Oral Efficacy in the Preformed Fibrils (PFF) Mouse Model of Parkinson's Disease

Objective

To determine the oral efficacy of the compound of formula (I) in a chronic progressive model of Parkinson's disease (PD), the preformed fibrils (PFF) mouse model, using a prophylactic and therapeutic dosing schedule.

Treatment

8-week-old C57BL6 male mice (obtained from ARC, Perth, Australia) were housed under a 12-h light cycle in a SPF climate-controlled facility with food and water provided ad libitum for two weeks prior to study initiation. The compound of formula (I) (or water alone for control animals) was administrated to mice in drinking water at 0.3 mg/ml. Animals were separated into the groups described in Table 9, with ten animals initiated in each cohort. For prophylactic dosing, treatment commenced one day prior to PFF-Synuclein injection. For therapeutic dosing, treatment commenced 4 months after disease induction with PFF-Synuclein.

TABLE 9
Study design for PFF-Synuclein model

| | Commencement | Behavioural | Bioanalysis |
|---|---|---|---|
| | of treatment or | readouts | (Dopamine, |
| | water (control) | (rotarod | DOPAC, |
| | versus injection | and balance | HVA and IL- |
| Group | time | beam) | 1β) |
| Group 1: animals | −24 hours (water) | 4, 6, 8 and | 12 months |
| injected with saline | | 10 months | (termination) |
| Group 2: animals | −24 hours (water) | 4, 6, 8 and | 12 months |
| injected with PFF- | | 10 months | (termination) |
| Synuclein | | | |
| Group 3: animals | −24 hours | 4, 6, 8 and | 12 months |
| injected with PFF- | (compound of | 10 months | (termination) |
| Synuclein and treated | formula (I), | | |
| prophylactically | 0.3 mg/ml) | | |
| Group 4: animals | 4 months after | 4, 6, 8 and | 12 months |
| injected with PFF- | injection | 10 months | (termination) |
| Synuclein and treated | (compound of | | |
| therapeutically | formula (I), | | |
| | 0.3 mg/ml) | | |

Preparation of Fibrillar α-synuclein

Recombinant human a-synuclein was obtained from rPeptide Inc., and in vitro fibril generation was performed with a final concentration of 2 mg/ml in phosphate-buffered saline (PBS) by incubation at 37° C. with agitation in an orbital mixer (400 rpm) for 7 days with daily cycles of sonication used to break down fibrillar aggregates as outlined in previously published reports (see Luk et al., Science, 2012, vol. 338(6109), pp. 949-53; and Zhang et al., Methods Mol. Biol., 2019, vol. 1948, pp. 45-57, which are incorporated in their entirety herein by reference). The generation of fibrillar α-synuclein species was confirmed by transmission electron microscopy and Thioflavin T fluorescence prior to use.

Surgical Procedure

Mice were anaesthetized using ketamine (100 mg/kg, i.p.) and xylazine (10 mg/kg i.p.) anesthesia, and were placed into a stereotactic frame with nose and ear bars specially adapted for mice. The lesion was performed using a 5 μl Hamilton syringe to deliver either vehicle or human PFF-Synuclein (8 μg) at the following coordinates relative to bregma: AP: +0.5 mm; ML: −2.0 mm; DV: −3.0 mm into the right dorsal striatum according to the stereotaxic atlas (“The mouse brain in stereotaxic coordinates” by Paxinos and Franklin, 1997). After drilling a 1 mm burr-hole in the skull, a 2 μl volume of solution was infused at the target site at the rate of 0.2 μl per minute. The needle was held in place for at least 5 minutes after injection to minimize retrograde flow along the needle tract. Mice were administered a subcutaneous injection of sterile Ringer's solution to facilitate recovery and were placed on a heat-pad until complete recovery from anesthesia.

Behavioural Tests

All behavioural tests were performed during the light phase of the light/dark cycle. Prior to each test, the mice were moved to the testing room for an acclimatization period of at least 30 min. Instruments and tools used for the behavioural tests were cleaned thoroughly with 70% ethanol and rinsed with sterile water between trials to minimize odours.

Balance Beam Test

Mice were tested on a 0.5 cm wide, im long balance beam apparatus. The balance beam consisted of a transparent Plexiglas structure that was 50 cm high with a dark resting box at the end of the runway. Mice were trained on the beam three times in the morning, allowing for a resting inter-trial period of at least 15 min. Mice were left in the dark resting box for at least 10 s before being placed back in their home cage. Mice were then re-tested in the afternoon, at least 2 h after the training session. During test sessions, mice performance was recorded. The test consisted of three trials with a resting inter-trial period of at least 10 min. The latency to cross the beam was recorded for the last of the three tests. Mice were tested, at 4, 6, 8 and 10 months after PFF or vehicle injection.

Rotarod Test

The accelerated rotarod test was performed over 3 consecutive days allowing for 2 days of training and acclimatization. Three trials per day were performed using a Rotarod (Ugo Basile) apparatus with an accelerated speed of 5-40 RPM in 5 min. A resting time of at least 30 min was given between trials. Latency to fall was recorded at each time. Every mouse able to stay on the rotating rod for more than 5 min was removed and its latency recorded as 300 s. The average of the 3 trials performed is presented. Mice were tested, at 4, 6, 8 and 10 months after PFF or vehicle injection.

IL-1β ELISA for Plasma Determination

The mouse IL-1β kit (R&D Systems, Catalog #DY008), was used to measure IL-1β concentration in plasma samples from mice at culling time point (12 months). Plasma samples were diluted 1 in 5 following the manufacturer instructions.

LC-MS/MS Quantification of Striatal Dopamine and Metabolites

At 12 months mice were sacrificed and striatal tissue was micro-dissected, weighed and snap-frozen at −80° C. Neurotransmitters from striatal tissues were extracted and derivatized using ethyl chloroformate. Striatal dopamine (DA) and its metabolites (DOPAC and HVA) were quantified in their stable derivative form in the presence of internal standard 3,4-dihydroxybenzylamine (DHBA) using highly sensitive liquid chromatography-tandem mass spectrometry (LC-MS/MS) as described previously (Park et al., Biol. Pharm. Bull., 2013, vol. 36(2), pp. 252-8). An API 3200 (AB SCIEX) triple quadrupole Q TRAP LC/MS/MS system was used with Turbo V ion source coupled with Agilent series HPLC system under positive (+1) ionization in multi-reaction mode. Samples were chromatographed on a Phenomenex synergi fusion—RP 80 Å analytical column (150×4.6 mm; 4 μm) under a binary gradient condition at 500 μl flow rate using mobile phase A (0.1% formic acid in milliQ water) and mobile phase B (0.1% formic acid in acetonitrile). For quantitation, one transition per analyte was monitored and two transitions per analyte were monitored for qualitative purposes.

Results

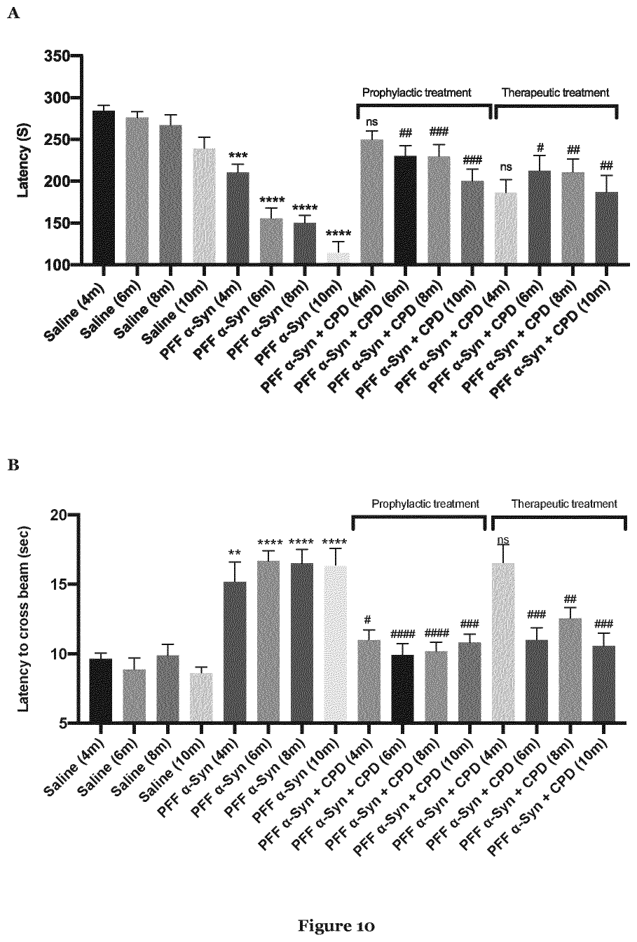

The behavioural results are shown in FIG. 10. PFF-Syn mice develop progressive motor deficits and loss of striatal dopamine with significative changes 4 months after injection. Drinking water administration of the compound of formula (I) (0.3 mg/ml) from 24 h before PFF injection (Prophylactic) and from 4 months after PFF injection (Therapeutic) both resulted in improved performance on the rotarod (FIG. 10A). Similar results were obtained with the balance beam test (FIG. 10B).

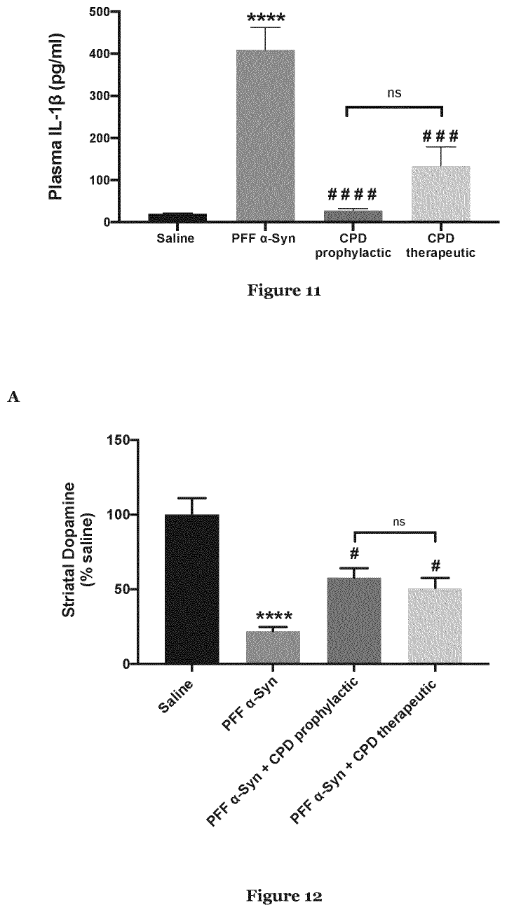

In order to characterize the profile of circulating IL-1β in plasma, IL-1β was measured through ELISA at 12 months after the PFF-Synuclein injections. The results shown in FIG. 11 demonstrate that both prophylactic and therapeutic treatments resulted in a significant decrease in circulating IL-1β compared with the PFF-Synuclein group. The decrease observed for the prophylactic treatment with the compound of formula (I) was greater than that observed with the therapeutic dosing groups.

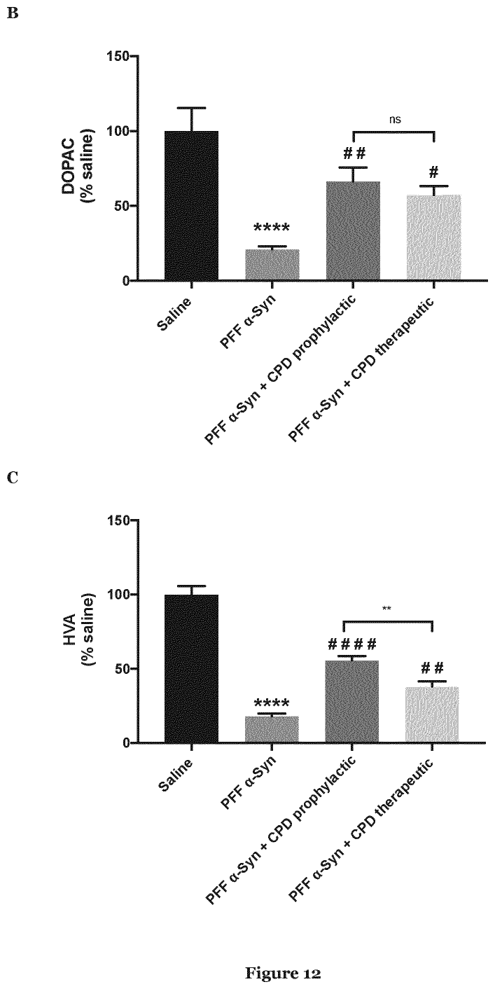

The results of the analysis of the levels of striatal dopamine (DA) and its metabolites (DOPAC and HVA) are shown in FIG. 12.

Previous studies using the PFF model have shown that there is a progressive loss of dopaminergic neurons in the substantia nigra, accompanied by a reduction in striatal dopamine on the injected side (see Zhang et al., Methods Mol. Biol., 2019, vol. 1948, pp. 45-57; and Gordon et al., Sci. Transl. Med., 2018, vol. 10(465), which are incorporated in their entirety herein by reference). Consistent with these reports, in untreated PFF-syn mice, a substantial reduction in striatal dopamine and dopamine metabolites at 12 months was observed. In contrast, PFF-syn mice treated with the compound of formula (I) in both prophylactic and therapeutic settings had significantly higher striatal dopamine concentrations (P<0.02 and P<0.05 respectively; FIG. 12A), indicating that this drug protected against dopaminergic degeneration induced by α-synuclein pathology. The treated mice also had significantly higher dopamine metabolites DOPAC (FIG. 12B) and HVA (FIG. 12C) compared to the untreated PFF mice.

Accordingly, it can be concluded that oral administration of the compound of formula (I) in the PFF mouse model of Parkinson's disease results in effective treatment in both a therapeutic and prophylactic capacity. The prophylactic treatment group in particular showed excellent efficacy towards motor impairment and dopamine loss. Notably, the therapeutic treatment group, starting at 4 months after PFF-Synuclein injections, also is showed a significant improvement in motor function, compared to the PFF-Synuclein group, indicating that treatment with the compound of formula (I) can arrest further dopaminergic degeneration in the model. Interestingly, the measurement of dopamine and their metabolites at the end of the experiment (12 months) demonstrated neuroprotection in both prophylactic and therapeutic treatment regimes. This suggests that despite apparent motor deficits at 4 months, the compound of formula (I) can still prevent further decline of dopamine loss. The results provide a strong indication that treatment in Parkinson's disease patients, especially those with active motor symptoms and/or dopamine loss, will prove beneficial.

1-21. (canceled)

22. A method for the treatment or prevention of a neurodegenerative disorder in a patient in need thereof, wherein the method comprises administering to the patient in need thereof a therapeutically or prophylactically effective amount of a compound of formula (I): 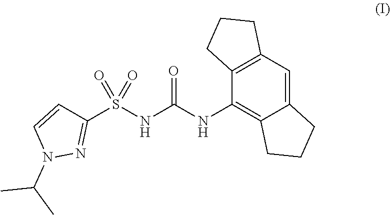 [SMILES: CC(C)n1ccc(S(=O)(=O)NC(=O)Nc2c3c(cc4c2CCC4)CCC3)n1]
or a pharmaceutically acceptable salt thereof.

23. The method as claimed in claim 22, wherein the neurodegenerative disorder is Parkinson's disease.

24. The method as claimed in claim 22, wherein the neurodegenerative disorder is Alzheimer's disease.

25. The method as claimed in claim 22, wherein the neurodegenerative disorder is Motor Neurone disease.

26. The method as claimed in claim 22, wherein the neurodegenerative disorder is Huntington's disease.

27. The method as claimed in claim 22, wherein the neurodegenerative disorder is Multiple System Atrophy.

28. The method as claimed in claim 22, wherein the neurodegenerative disorder is Progressive Supranuclear Palsy.

29. The method as claimed in claim 22, wherein the neurodegenerative disorder is Frontotemporal Dementia.

30. The method as claimed in claim o, wherein the neurodegenerative disorder is Ataxia.

31. The method as claimed in claim 22, wherein the neurodegenerative disorder is a Neurodegenerative Prion Disease.

32. The method as claimed in claims 22, wherein the treatment or prevention comprises the treatment or prevention of neuroinflammation.

33. The method as claimed in claim 22, wherein the treatment or prevention comprises the oral administration of the compound or the salt thereof.

34. The method as claimed in claim 22, wherein the compound or salt is a sodium salt such as a monosodium salt.

35. (canceled)

36. The method as claimed in claim 22, wherein the compound or salt is a monohydrate.

37. The method as claimed in claim 22, wherein the compound or salt is crystalline.

38. The method as claimed in claim 22, wherein the compound or salt is a crystalline monosodium monohydrate salt.

39. The method as claimed in claim 38, wherein the crystalline monosodium monohydrate salt has an XRPD spectrum comprising peaks at: 4.3°2θ, 8.7°2θ, and 20.6°2θ, all ±0.2°2θ.

40. The method as claimed in claim 38, wherein the crystalline monosodium monohydrate salt has an XRPD spectrum in which the 10 most intense peaks include 5 or more peaks which have a 2θ value selected from: 4.3°20, 6.2°2θ, 6.7°2θ, 7.3°2θ, 8.7°2θ, 9.0°2θ, 12.1°2θ, 15.8°2θ, 16.5°2θ, 18.0°2θ, 18.1°2θ, 20.6°2θ, 21.6°2θ, and 24.5°2θ, all ±0.2°2θ.

41. The method as claimed in claim 22, wherein the compound or the pharmaceutically acceptable salt thereof is administered as a pharmaceutical composition further comprising a pharmaceutically acceptable excipient.

42. The method as claimed in claim 41, wherein the pharmaceutical composition is suitable for oral administration.
